(19) United States
(12) Patent Application Publication
(10) Pub. No.: US 2007/0106358 A1
(43) Pub. Date: May 10, 2007

(54) Tissue stimulating lead and method of implantation and manufacture

(75) Inventors: Randy W. Westlund, River Falls, WI (US); Paul E. Zarembo, Vadnais Heights, MN (US)

Correspondence Address: FAEGRE & BENSON, LLP;BOSTON SCIENTIFIC PATENT DOCK, 2200 WELLS FARGO CENTER, 90 SOUTH SEVENTH STREET, MINNEAPOLIS, MN, 55402-3901, US

(73) Assignee: Cardiac Pacemakers, Inc., St. Paul, MN (US)

(21) Appl. No.: 11/266,958

(22) Filed: Nov. 4, 2005

(51) Int. Cl. A61N 1/00

(52) U.S. Cl. 607/122

(57) ABSTRACT

An injectable cardiac lead for stimulating a heart includes an anchor and a tether. The anchor has first and second electrically active areas spaced apart and electrically isolated from one another. The tether has a distal end mechanically coupled to the anchor and a proximal end adapted for coupling to a cardiac rhythm management device. The tether comprises first and second electrically conductive cables electrically isolated from one another and electrically coupled to the first and second electrically active areas, respectively.

### TECHNICAL FIELD

The present invention relates to medical devices and methods for accessing an anatomical space of the body. More specifically, the invention relates to minimally-invasive devices and methods for implanting an electrode in a myocardium of a heart.

### BACKGROUND

Cardiac rhythm management systems are used to treat heart arrhythmias. Pacemaker systems are commonly implanted in patients to treat bradycardia (i.e., abnormally slow heart rate). A pacemaker system includes an implantable pulse generator and leads, which form the electrical connection between the implantable pulse generator and the heart. An implantable cardioverter defibrillator (“ICD”) is used to treat tachycardia (i.e., abnormally rapid heart rate). An ICD also includes a pulse generator and leads that deliver electrical energy to the heart.

The leads coupling the pulse generator to the cardiac muscle are commonly used for delivering an electrical pulse to the cardiac muscle, for sensing electrical signals produced in the cardiac muscle, or for both delivering and sensing. The leads are susceptible to categorization according to the type of connection they form with the heart. An endocardial lead includes at least one electrode at or near its distal tip adapted to contact the endocardium (i.e., the tissue lining the inside of the heart). An epicardial lead includes at least one electrode at or near its distal tip adapted to contact the epicardium (i.e., the tissue lining the outside of the heart). Finally, a myocardial lead includes at least one electrode at or near its distal tip inserted into the heart muscle or myocardium (i.e., the muscle sandwiched between the endocardium and epicardium). Some leads have multiple spaced apart distal electrodes at differing polarities and are known as bipolar type leads. The spacing between the electrodes can affect lead performance and the quality of the electrical signal transmitted or sensed through the heart tissue.

The lead typically consists of a flexible conductor surrounded by an insulating tube or sheath that extends from the electrode at the distal end to a connector pin at the proximal end. Endocardial leads are typically delivered transvenously to the right atrium or ventricle and commonly employ tines at a distal end for engaging the trabeculae.

The treatment of congestive heart failure (“CHF”), however, often requires left ventricular stimulation either alone or in conjunction with right ventricular stimulation. For example, cardiac resynchronization therapy (“CRT”) (also commonly referred to as biventricular pacing) is an emerging treatment for heart failure, which requires stimulation of both the right and the left ventricle to increase cardiac output. Left ventricular stimulation requires placement of a lead in or on the left ventricle in the lateral or posterior-lateral aspect/region of the heart. One technique for left ventricular lead placement is to expose the heart by way of a thoracotomy. The lead is then positioned so that the electrodes contact the epicardium or are embedded in the myocardium. Another method is to advance an epicardial lead endovenously into the coronary sinus and then advance the lead through a lateral vein of the left ventricle. The electrodes are positioned to contact the epicardial surface of the left ventricle.

The epicardium is a tough, fibrous tissue layer. It may be difficult to penetrate the epicardium when manipulating a tool from a distance, as when minimally invasive surgical techniques are used. In addition, repeated or failed attempts to penetrate the epicardium may result in increased trauma to the heart. Furthermore, the left ventricle beats forcefully as it pumps oxygenated blood throughout the body. Repetitive beating of the heart, in combination with patient movement, can sometimes dislodge the lead from the myocardium. The electrodes may lose contact with the heart muscle, or spacing between electrodes may alter over time.

There is a need for an improved pacing lead suitable for chronic implantation and a minimally invasive delivery system and method for implanting such a lead into the myocardium.

### SUMMARY

In one embodiment, the present invention is a cardiac lead for stimulating a myocardium of a heart. The cardiac lead includes an anchor and a tether. The anchor has first and second electrically active areas spaced apart and electrically isolated from one another. The tether has a distal end mechanically coupled to the anchor and a proximal end adapted for coupling to a cardiac rhythm management device. The tether includes first and second electrically conductive cables electrically isolated from one another and electrically coupled to the first and second electrically active areas, respectively.

In another embodiment, the present invention the present invention is a cardiac lead for stimulating a myocardium of a heart. The cardiac lead includes an anchor and a tether. The anchor has a first electrically active area. The tether has a distal end mechanically coupled to the anchor and a proximal end adapted for coupling to a cardiac rhythm management device. The tether includes a first electrically conductive cable electrically isolated coupled to the first electrically active area. An outer diameter of the tether from the proximal end to the distal end is substantially isodiametric.

In yet another embodiment, the present invention is a method of implanting a cardiac lead into a myocardium of a heart, wherein the lead includes an anchor and a tether. The anchor has first and second electrically active areas spaced apart and electrically isolated from one another. The tether has a distal end mechanically coupled to the anchor and a proximal end adapted for coupling to a cardiac rhythm management device. The tether includes first and second electrically conductive cables electrically isolated from one another and electrically coupled to the first and second electrically active areas, respectively. The anchor is engaged to a distal end of an insertion tool and the distal end of the insertion tool is advanced to the heart. The anchor is injected into a myocardium of the heart by ejecting the anchor from the insertion tool so that the first and second electrically active areas are located within the myocardium. The tool is withdrawn proximally and the anchor is deployed to a configuration adapted to resist migration by tensioning the tether.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 shows an exemplary implantable medical device including a lead attached to a heart according to an embodiment of the present invention.

FIG. 2 shows a cross-sectional view of the lead of FIG. 1.

FIG. 3 shows a cross-sectional view of a distal end of a lead according to another embodiment of the invention in which the tether is coupled closer to a first end of the anchor than a second end.

FIG. 4 shows a cross-sectional view of a distal end of a lead according to another embodiment of the invention in which the anchor is provided with a notch to facilitate removal.

FIG. 5 shows a cross-sectional view of a distal end of a lead according to another embodiment of the invention in which the anchor has a curved configuration.

FIG. 6 shows a side plan view of a distal end of a lead according to another embodiment of the present invention.

FIG. 7 shows a perspective view of a distal end of a lead according to another embodiment of the invention in which the lead is adapted for unipolar use.

FIG. 8 shows a perspective view of a distal end of a lead according to another embodiment of the present invention in which the electrodes are located proximally from the anchor.

FIG. 9A shows a perspective view of a distal end of a lead according to another embodiment of the invention.

FIG. 9B shows a perspective view of a distal end of a lead according to another embodiment of the invention.

FIG. 10A shows a cross-sectional view of a distal end of a lead in a collapsed configuration according to another embodiment of the invention.

FIG. 10B shows a cross-sectional view of the distal end of the lead of FIG. 10A in an expanded configuration.

FIG. 11 shows a cross-sectional view of a distal end of a lead according to another embodiment of the invention.

FIG. 12 shows a cross-sectional view of a distal end of a lead according to another embodiment of the invention.

FIG. 13 shows a lead including a retention feature in relation to the anatomical layers of the heart according to an embodiment of the invention.

FIG. 14A shows a cross-sectional view of a lead and a tool for injecting the lead into the heart in relation to the anatomical layers of the heart according to an embodiment of the invention.

FIG. 14B shows a cross-sectional view of the lead of FIG. 14A deployed within the heart.

FIG. 15A shows a cross-sectional view of a tool for injecting a lead into the heart according to another embodiment of the present invention.

FIG. 15B shows a cross-sectional view of the tool of FIG. 15A taken along line B-B.

FIG. 16 shows a cross-sectional view of a tool for injecting a lead into the heart according to another embodiment of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

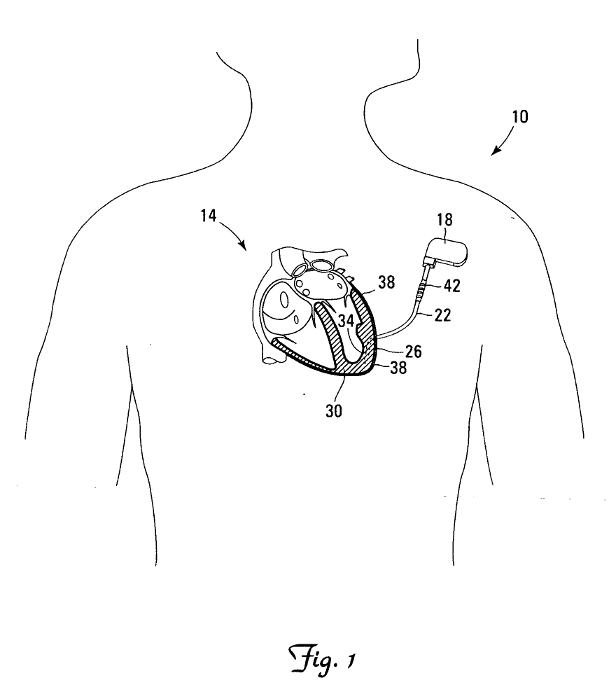

FIG. 1 illustrates an implantable medical device 10 for sensing or pacing of a heart 14. The implantable medical device 10 includes a cardiac rhythm management device such as a pulse generator 18 implanted in the chest or abdomen and a lead 22. The lead 22 has a distal end 26 implanted in a myocardium 30 of the heart 14 between an inner endocardium 34 and an outer epicardium 38. A proximal end 42 of the lead 22 is electrically couplable to the pulse generator 18.

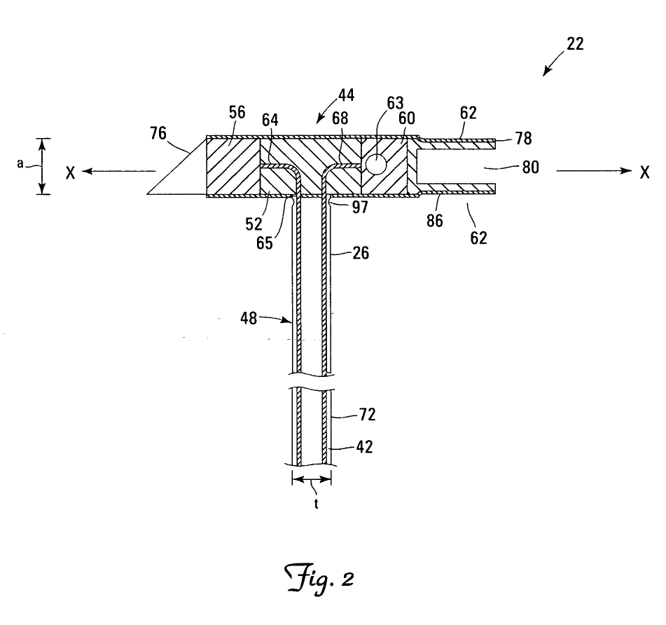

FIG. 2 shows the lead 22 in greater detail according to one embodiment of the present invention. The lead 22 includes a distal tissue anchor 44 coupled to a flexible tether 48. The anchor 44 serves as an electrode for sensing or pacing the heart 14, while electrical signals are transmitted to and from the pulse generator 18 via the tether 48. The anchor 44 may take many different configurations suitable for implantation into the heart 14. In general, however, the lead 22 has a reduced outer diameter at both the anchor 44 and tether 48 as would permit injection of the lead 22 into the heart 12.

In one embodiment, the anchor 44 is deployable from a first, collapsed configuration to a second, expanded configuration. By collapsed it is meant that the anchor 44 has a profile sized, shaped and otherwise adapted to traverse the myocardium 30 with minimal trauma thereto in conjunction with a minimally invasive surgical procedure. By expanded, it is meant that the anchor 44 has a profile configured to resist traversing the myocardium 30 and migrating or displacing from an implantation site within the heart 14. In some embodiments, the anchor 44 is merely repositioned when deploying from the first configuration to the second configuration. In other embodiments, the size and/or shape of the anchor 44 changes when deploying from the first configuration to the second configuration.

The anchor 44 is sized and shaped to anchor or fix the electrodes 56, 60 in the heart 14, such that when the anchor 44 is implanted in the heart 14, the-electrodes 56 and 60 are in contact with the heart 14. In one embodiment, as is shown in FIG. 2, the anchor 44 is elongated and shaped like a bar or cylinder having a longitudinal axis X. However, the anchor 44 may take many shapes, examples of which are described further below.

The anchor 44 as shown includes a separation member 52 formed of a non-conductive material which electrically isolates a first electrically active surface or electrode 56 of the anchor 44 from a second electrically active surface or electrode 60 of the anchor 44. In the present embodiment, the lead 22 is capable of bipolar pacing and sensing in which one of the electrodes 56, 60 is an anode and the other is a cathode. In other embodiments, however, the lead 22 includes a single electrode 56 for unipolar pacing and sensing. Alternately, the lead 22 includes a greater number of electrodes. The electrodes may be positioned on the lead 22 to stimulate or sense different layers of the heart 12. In one embodiment, for example, the lead 22 includes four electrodes (not shown). The electrodes 56, 60 may also be referred to as electrically active areas or surfaces.

The body of the anchor 44 may be formed of a variety of biocompatible materials, including silicon rubber and polyurethane, and may be rigid or flexible. In one embodiment, the separation member 52 is formed of a non-conductive tubular structure, for example, PEEK (polyetheretherketone) tubing. The electrodes 56, 60 may be held in place adjacent the separation member 52 by variety of attachment means, including crimping, welding/fusing techniques, and adhesives, including, for example, epoxy, polyurethane, and silicone adhesives.

The anchor 44 is sized to be as small as possible yet still have a sufficient surface area at the electrodes 56 and 60 to make contact with the heart 14 for efficient sensing and pacing. In one embodiment, the anchor 44 has an outer diameter a of from about 1 French to about 6 French (0.333 to about 2 mm). In another embodiment, the outer diameter a of the anchor 44 is from about 1.5 French to about 2.5 French (0.5 to about 0.833 mm). In one embodiment, each electrode 56, 60 has a length of from about 0.5 mm to about 1.5 mm. In another embodiment, each electrode has a length of about 1 mm. In one embodiment, the electrodes 56, 60 are spaced apart from one another by from about 5 mm to about 9 mm. In another embodiment, the electrodes 56, 60 are spaced apart from one another by about 7 mm.

The electrodes 56, 60 may include surface treatments, coatings or other means for minimizing charge injection, maximizing sensing capabilities and/or helping to reduce pacing thresholds. Such surface treatments or coatings may include IROX (iridium oxide-coated titanium) or other compatible treatments. The electrodes 56, 60 may also be provided with a surface treatment or other means to increase electrode surface area.

In one embodiment, at least a portion of the anchor 44 is provided with a coating 62 of a therapeutic treatment or drug such as a steroid. The treatment or drug is thus delivered to the tissues of the heart 14 in the immediate locale in which sensing and pacing occurs, and also in the immediate locale in which the heart 14 may suffer trauma during implantation. Such steroids or other therapeutic treatments can therefore more effectively reduce inflammation and/or provide drug therapy.

In one embodiment, at least a portion of the anchor 44 is formed of a material chosen to reduce tissue ingrowth. One such exemplary material is ePTFE (expanded polytetrafluoroethylene). Reduced tissue ingrowth facilitates removal and repositioning of the lead 22. In another embodiment, a portion of the anchor 44 is formed of a material chosen to enhance tissue ingrowth. Enhanced tissue ingrowth may provide improved fixation to the heart 14 and thus enhanced lead stability. In another embodiment, the anchor 44 is formed with features to facilitate mechanical attachment of the lead 22 to the heart 14. For example, as is shown in FIG. 2, a channel 63 may extend through the anchor 44. Following implantation, tissue will tend to invade the channel 63, increasing fixation of the anchor 44 to the heart 14. In other embodiments, the anchor 44 may be provided with tines or ridges increasing the amount of surface area to which tissue may attach (not shown).

The tether 48 is formed of an electrically conductive material and thereby communicates electrical signals from the pulse generator 18 to the electrodes 56 and 60 and vice versa. The proximal end 42 of the lead 22 and tether 48 is adapted for coupling to the pulse generator 18. The distal end 26 of the tether 48 is mechanically coupled to the anchor 44 and electrically coupled to the electrodes 56 and 60. This may be accomplished in a variety of ways. In one embodiment, as is shown in FIG. 2, the separation member 52 is provided with an access aperture 65 through which the tether 48 passes. This allows connection of the tether 48 to the electrodes 56, 60 inside of the anchor 44. In other embodiments, the tether 48 is coupled to the anchor 44 and electrodes 56, 60 by, for example, crimping the tether 48 to the anchor 44 adjacent the electrodes 56, 60.

The tether 48 is coupled to the anchor 44 between a first end 76 of the anchor 44 and a second end 78 of the anchor 44. In this configuration, tension exerted on the tether 48 will tend to rotate or swing the anchor 44 from the first configuration in which anchor axis X is aligned with the tether 48 to the second configuration in which the anchor axis X is transverse to the tether 48. This tendency facilitates anchoring of the anchor 44 in the myocardium 30 or against a surface such as the endocardium 34 or the epicardium 38.

In the present embodiment, the tether 48 is formed of first and second cables 64 and 68 electrically isolated from one another and electrically coupled to the first and second electrodes 56 and 60, respectively. The first and second cables 64, 68 may be separate, or, as is shown in FIG. 2, may be encased in an outer sheath 72 such that the tether 48 has a unitary construction. In one embodiment, the cables 64 and 68 are twisted cables. Twisted cables may have a reduced size and mass in comparison to the more common spiral- or coiled-type leads.

In one embodiment, the tether 48 has an outer diameter t of from about 0.8 French to about 3 French (0.267 to about 1 mm). In another embodiment, the outer diameter t of the tether 48 is about 1.5 French (0.5 mm). In one embodiment, as shown in FIG. 2, the outer diameter t of the tether 48 is no greater than one half of the outer diameter of the tether 48. In one embodiment, as shown in FIG. 2, the outer diameter t of the tether 48 is substantially isodiametric, or has substantially the same outer diameter, along the length of the tether 48 from the distal end 26 of the tether 48 adjacent the anchor 44 to the proximal end 42 of the tether 48 (excluding any features provided on the proximal end of the tether 48 for coupling the lead 22 to a cardiac rhythm management device (not shown). Thus, substantially the entire lead 22 has a reduced outer diameter.

A lead having a reduced outer diameter provided several benefits. For example, the size of any passageway through the tissues of the heart 14 formed by the lead 22 is reduced, potentially reducing trauma to the patient during insertion. A lead 22 having a reduced outer diameter has a smaller outer surface area which reacts to the body, potentially reducing the formation of scar tissue about the lead 22. A lead 22 having a reduced outer diameter, and in particular having a cable construction as shown in the accompanying figures has a reduced mass. Reduced mass, in conjunction with reduced outer diameter, provides a lead which has a reduced impact on blood flow and which is impacted less by blood flow. The lead 22 may be advanced into smaller vessels during implantation and may be implanted using smaller, less invasive tools in conjunction with minimally invasive surgical procedures. Tether 48 may also be more flexible because of the reduced outer diameter and simplified construction.

The cables 64 and 68 may be formed of a variety of conductive materials, including platinum-clad tantalum. In one embodiment, as is shown in FIG. 2, the cables 64, 68 include insulation, for example, a non-conductive coating so that the cables 64, 68 are electrically isolated from one another and from the patient's tissues. In other embodiments, however, the cables 64 and 68 may be positioned in isolated lumens of the sheath 72 or may be provided with other means for electrically isolating from one another and from the patient's tissues.

The anchor 44 may include features to facilitate implantation into the heart 14. In one embodiment, a first end 76 of the anchor 44 is pointed and/or has a sharpened edge to dissect tissue. In other embodiments, the first end 76 of the anchor 44 is blunt or a combination of blunt and pointed (i.e., bullet shaped). The first end 76 of the anchor 44 may be shaped to puncture or pierce the epicardium 38 in such a way that trauma to the epicardium 38 and the amount of force needed to puncture or pierce the epicardium 38 is reduced.

A second end 78 of the anchor 44 may be adapted to cooperate with a tool maneuverable to implant the lead 22 in the heart 14. In one embodiment, the anchor 44 includes a first tool engaging area 80 including a recess extending into the second end 78 of the anchor 44. The second end 78 of the anchor 44 may also include a second tool engaging area forming a shoulder 86. The tool engaging shoulder 86 may be sized and shaped such that the second end 78 of the anchor 44 can be inserted into the distal end of a tool. The anchor 44 may also include other types of tool engaging features, including, for example, surface irregularities, grooves, recesses, ridges, threads or other means for grasping by insertion tools.

It may be desirable to remove or revise the lead 22 after implantation. In one embodiment, the lead 22 includes a detachment means for detaching the distal end 26 of the tether 48 from the anchor 44. In one embodiment, the tether 48 has a weak region 97 proximally adjacent to the anchor 44. Upon the exertion of sufficient tension on the tether 48 by the surgeon, the weak region 97 fails and the tether 48 separates from the anchor 44, leaving the anchor 44 safely encapsulated within the myocardium 30.

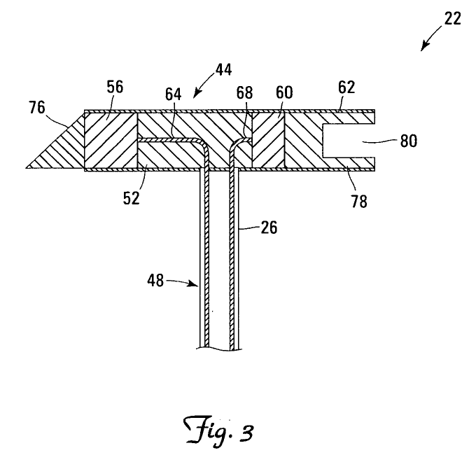

In one embodiment, as is shown in FIG. 3, the connection between the anchor 44 and tether 48 is modified to facilitate detaching the tether 48 from the anchor 44 after implantation. Instead of or in addition to the inclusion of weak region 97 as is shown in FIG. 2, the tether 48 is coupled to the anchor 44 closer to the second 78 than the first end 76, or vice versa. The tether 48 is still positioned relative to the first and second ends 76 and 78 such that tension exerted on the tether 48 tends to deploy the anchor 44 from the first configuration to the second configuration (i.e., the anchor is rotated from a longitudinally oriented position to a transverse position). The tension, however, is transferred from the tether 48 to the anchor 44 slightly unevenly, reducing the minimum tension required to separate the tether 48 from the anchor 44.

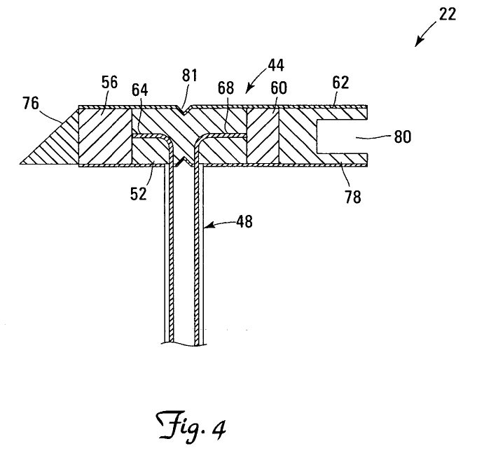

In one embodiment, as is shown in FIG. 4, the detachment means is a notch 81 is formed in the anchor 44 between the first and second ends 76, 78 and aligned with connection of the tether 48 to the anchor 44. The notch 81 provides a fold or stress line to facilitate removal or revision of the anchor 44. Upon sufficient tension being exerted on the tether 48, the anchor 44 will tend to buckle along the notch 81, allowing for easier removal of the anchor 44 from the heart 14.

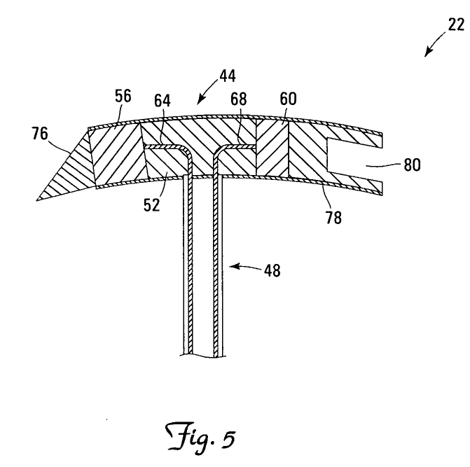

In one embodiment, as is shown in FIG. 5, the anchor 44 is curved. The degree of curvature may be chosen to facilitate insertion through the tissues of the heart 14. The degree of curvature may also be chosen to conform to the geometry of a surface of the heart 14.

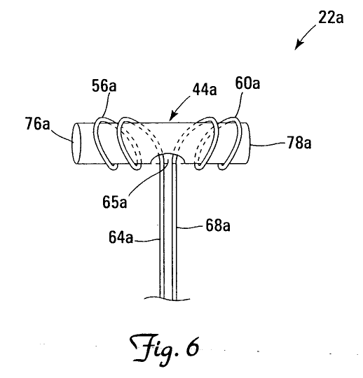

FIGS. 6-12 show the lead 22 according to various embodiments of the present invention. Similar parts in relation to the embodiments shown in FIGS. 2-5 are given similar numbering with the addition of “a”, “b”, “c”, etc. In general, a lead according to the present invention may employ any combination of some or all of the features discussed herein, and the invention is not limited to any particular combination as shown or discussed. In each of these embodiments, as further discussed below, the anchor 44 may be implanted in the myocardium 30 or positioned to engage the epicardium 38 or the endocardium 34.

FIG. 6 shows a lead 22a according to another embodiment of the present invention. As is shown in FIG. 6, the cables 64a, 68a may first be threaded through an aperture 65a in the anchor 44a and then coiled around opposite ends 76a and 78a of the anchor 44a. The distal tips of the cables 64a and 68a are secured to the anchor 44a to securely couple the tether 48a to the anchor 44a. The ends of the cables 64a, 68a which travel around the anchor 44a to form coils are exposed, thus serving as the electrodes 56a and 60a. A portion of the cables 64a, 68a thus form the electrodes 56a, 60a. Furthermore, the coils may be arranged to provide increased electrode surface area. Because the cables 64a and 68a are at least partially exposed, they should be formed of a biocompatible material. Exemplary materials include, but are not limited to, platinum-clad tantalum.

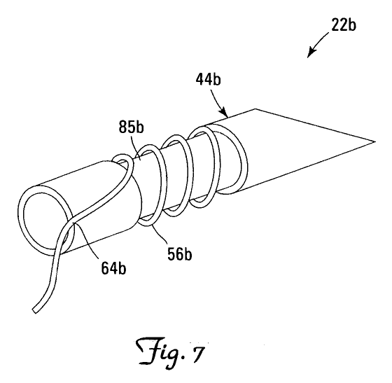

FIG. 7 shows a lead 22b according to another embodiment of the present invention. Lead 22b is similar to the lead 22a shown generally in FIG. 6 in that exposed coils the cable 64b forms an electrode 56b. In addition, the anchor 44b is provided with a recessed area 85b around which the exposed region of the cable 64b is coiled. Furthermore, lead 22b is configured as a unipolar lead and includes a single electrically active area or electrode 56b.

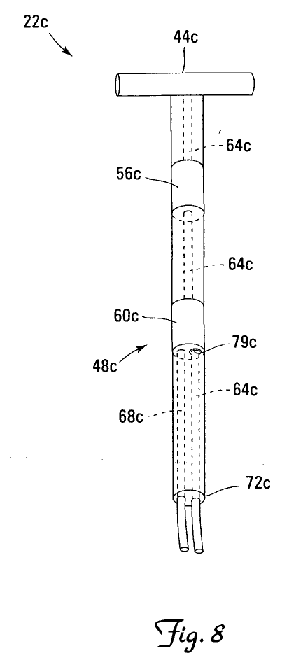

FIG. 8 shows a lead 22c according to another embodiment of the present invention. As is shown in FIG. 8, a first electrode 56c and a second electrode 60c are located on the tether 48c proximal to the anchor 44. The tether 48c includes a first cable 64c mechanically connected to the anchor 44c and electrically coupled to the first electrode 56c. The tether 48c further includes a second cable 68c electrically coupled to a second electrode 60c proximal to the first electrode 56c. The second electrode 60c includes a through-hole 79c through which the first cable 64c extends. The first and second cables 64, 68 are insulated so that the first cable 64c is not electrically coupled to the second electrode 60c as it passes through the through-hole 79c.

In one embodiment, as is shown in FIG. 8, an outer sheath 72c is disposed around the tether 48c proximal to the second electrode 60c and between the first and second electrodes 56c, 60c and between the first electrode 56c and the anchor 44c. The sheath 72c may also be mechanically coupled to the anchor 44c to improve the strength of the connection of the tether 48c to the anchor 44c. The anchor 44c may be deployed onto a surface of the heart 14 such as the endocardium 34 or the epicardium 38 or within the myocardium 30 such that the electrodes 56c, 60c are positioned within the myocardium 30. The spacing between the anchor 44c and the first electrode 56c and between the first electrode 56c and the second electrode 60c may be chosen to position the electrodes 56c and 60c within a particular region of the myocardium 30 or to accommodate a myocardium 30 having an unusual thickness.

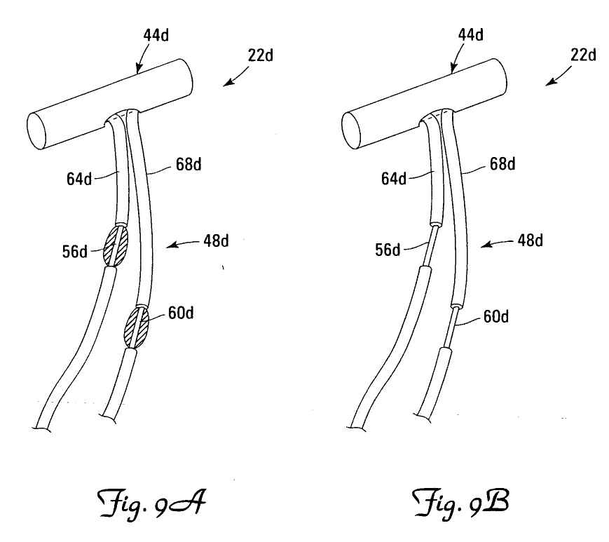

FIGS. 9A and 9B show a lead 22d according to another embodiment of the present invention in which electrodes 56d and 60d are located on the tether 48c proximal to the anchor 44c. In the present embodiment, the cables 64d, 68d that are electrically coupled to the respective electrodes remain separate from one another. The electrodes 56d and 60d may be coupled to the cables 64d and 68d, as shown in FIG. 9A. Alternately, as shown in FIG. 9B, the electrodes 56d and 60d may be no more than exposed regions of the cables 64d and 68d. The electrodes 56d and 60d are spaced apart from one another along the length of the respective cables 64d, 68d so as to avoid contacting one another and, if desired, to pace or sense different areas of the myocardium 30.

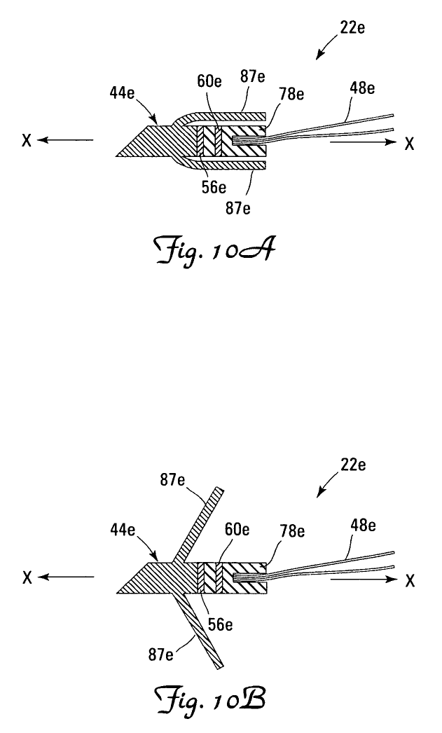

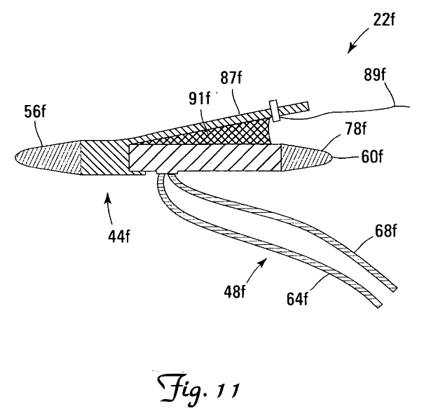

FIGS. 10A and 10B show a lead 22e according to another embodiment of the present invention. An anchor 44e of the lead 22e has at least a first movable wing or tab 87e, and optionally has two wings 87e as shown in FIGS. 10A and 10B. The wings 87e are deployable from a first configuration in which the wings 87e lie flat or are aligned with a longitudinal axis x of the anchor 44e, as is shown in FIG. 10A, to a second or expanded configuration in which the wings 87e are spread outwardly or away from the anchor 44e, as is shown in FIG. 10B. The tether 48e is coupled to a second end 78e of the anchor 44e such that upon tensioning the tether 48e in a proximal direction, the anchor 44e slides slightly proximally, causing the wings 87e to catch on the tissues of the heart 14 and deploy outwardly. When in the second configuration, the wings 87e prevent proximal migration of the anchor 44e within the heart 14. Electrodes 56e and 60e may be positioned on the anchor 44d proximal to the wings 87e so as to contact the heart 14 when the anchor 44e is deployed. In other embodiments, the electrodes 56e and 60e may be located on the wings 87e or on the tether 48e.

In other embodiments of the invention, the lead 22e may include other fixation means such as spines, barbs, tabs. These and any other fixation means may be provided in greater numbers and at different locations on the anchor 44e.

FIG. 11 shows a lead 22f according to another embodiment of the present invention in which the wings 87f are biased to an outwardly protruding configuration. The bias increases the likelihood of the anchor 44f successfully deploying to the second configuration. In one embodiment, the wings 87f are coupled to a tensioning device 89f extending proximally and accessible outside of the body following implantation of the lead 22. The tensioning device 89f may be coupled to one or more of the wings 87f such that tensioning of the tensioning device 89f causes the attached wings 87f to collapse to the first, collapsed configuration. The tensioning device 89f may be employed to reposition, re-deploy or remove the anchor 44f. The lead 22f may further include a webbing or mesh 91f attached to the anchor 44f and to the wing or wings 87f. The mesh 91f folds in on itself when the attached wings 87f are in the first collapsed configuration and stretch between the anchor 44f and the attached wings 87f when the anchor 44f is in the second, expanded configuration. When folded, the mesh 91f may reduce tissue ingrowth at the wing 87f, which might interfere with collapse of the wings 87f. The mesh could be made of, for example, rubber or ePTFE.

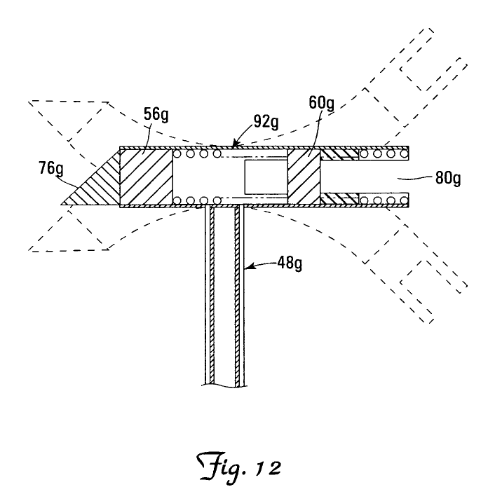

FIG. 12 shows a lead 22g according to another embodiment of the present invention in which the anchor 44g is adapted to be flexible. As shown in FIG. 12, the anchor 44g is constructed of an inner coiled member 90g located in an outer tube 92g. The coil structure provides flexibility to the anchor 44g to facilitate deployment and any subsequent repositioning of the anchor 44g. The anchor 44g further includes a tool-receiving recess 80g for receiving a tool, such as a stylet, to facilitate implantation. The tool may provide additional rigidity to the anchor 44g during implantation. The anchor 44g may also include other features as previously discussed, including a dissecting or cutting edge at a first end 76g of the anchor 44g, a pair of electrodes 56g and 60g, and a tether 48g.

Where the anchor 44 is intended to be located on a surface of the heart 14, such as the endocardium 34 or the epicardium 38, the lead 22 may also include means for chronically engaging the anchor 44 against the surface. This may be accomplished by fixing the tether 48 to the heart 14.

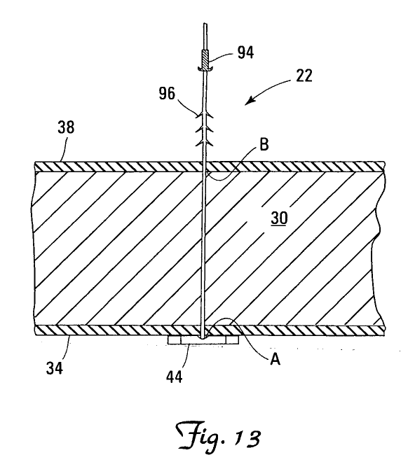

FIG. 13 shows the lead 22 implanted such that the anchor 44 engages the endocardium 34. The lead 22 is further provided with a retention feature 94 for fixing the anchor 44 into position. In the present embodiment, the retention feature 94 is slidable over the tether 48 so as to abut the epicardium 38. The retention feature 94 may have various configurations, including a button-like feature or a cylinder, and may be formed of various materials, including, for example, silicone rubber. The distal end of the tether 48 may include an engaging feature 96 such as tines or ridges for engaging the retention feature 94. The engaging features 96 facilitate a secure connection between the tether 48 and the retention feature 94 and reduce slippage between the tether 48 and the retention feature 94.

The retention feature 94 may be advanced over the tether 48 using a catheter or other tool. The tether 48 is tensioned and the retention feature 94 is fixed to the tether 48 adjacent the epicardium 38 to retain the portion of the tether 48 between points A and B in a tensioned state. The tension exerted by the tether 48 causes the anchor 44 to engage the endocardium 34 at point A, reducing lead migration.

The lead 22 may include other retention features or means for retaining the anchor 44 against a surface of the heart 12. For example, in other embodiments, the tether 48 may be sutured to a surface of the heart 12 to hold the anchor 44 tensioned against the heart 12. In still other embodiments, the lead 22 is pre-shaped to resist migration. For example, the distal end of the tether 48, i.e., that portion of the tether 48 that would be located within the myocardium 30, may be crimped, bent or otherwise shaped so as to resist migration (not shown).

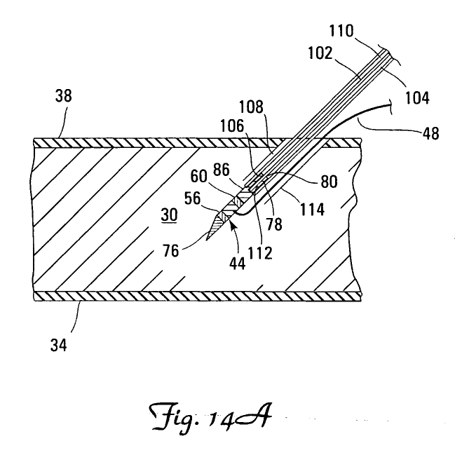

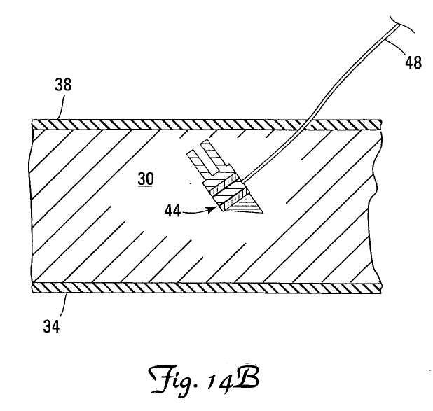

FIGS. 14A and 14B show another embodiment of the present invention, in which a tool 100 is provided for implanting the lead 22 into the heart 14. The tool 100 includes an injection mechanism 102 and an elongated tool body 104. The tool body 104 includes a nest 106 provided at a distal end 108 of the tool body 104 into which the anchor 44 is at least partially inserted. As previously described, the anchor 44 may include a tool engaging shoulder 86 or other means adapted for seating the anchor 44 within the nest 106. The tether 48 may extend outside of the tool body 104, as is shown in FIG. 14A, or may be threaded within a lumen 110 extending through the tool body 104 proximally from the nest 106.

The injection mechanism 102 may be a stylet or other plunger-like device that is inserted into the lumen 110 of the tool body 104 so that a distal end 112 is positioned proximally adjacent to the nest 106. The injection mechanism 102 may be inserted into the tool body 104 prior to or during the implantation procedure to provide the tool body 104 with additional support, or may be inserted into the tool body 104 later so that the tool body 104 remains flexible during the implantation procedure.

To implant the lead 22 into the heart 14, the anchor 44 is inserted into the nest 106. The distal end 108 of the tool body 104 is then advanced to the heart 14, using the first end 76 of the anchor 44 to cut or dissect the heart 14. The anchor 44 creates a tract 114 through the heart 14 within which the tool body 104 and the tether 48 extend proximally from the anchor 44.

When the anchor 44 has been placed in an appropriate site for sensing and pacing, the injection mechanism 102 is actuated to eject the anchor 44 from the nest 106 and inject the anchor 44 into the heart 14. In the present embodiment, the injection mechanism 102 is actuated when the surgeon slides the stylet distally through the lumen 110 relative to the tool body 104 and engages the anchor 44 at the tool engaging area 80. Doing so displaces the anchor 44 from the nest 106, injecting the anchor 44 into the heart 14. The tool 100 is then withdrawn proximally, leaving the anchor 44 in the implantation site and the tether 48 extending proximally through the tract 114. Tension may be exerted on the proximal end 42 of the tether 48 to deploy the anchor 44 from the first, collapsed configuration in which the anchor 44 is positioned longitudinally within the tract 114 to the second, or expanded configuration in which the anchor 44 is positioned transverse to the tract 114 (See FIG. 14B). The withdrawal of the tool 100 may be sufficient to tension the tether 48, particularly if the tether 48 is threaded through the tool body lumen 110. However, the surgeon may also manually tension the tether 48 by grasping the proximal end 42 of the tether 48 and pulling gently.

In the second configuration the anchor 44 is wider than the tract 114, helping to secure the anchor 44 in position and resist repositioning and migration. Following implantation of the anchor 44, the tether 48 is coupled to the pulse generator 18 to provide sensing and pacing of the heart 14 (See FIG. 1).

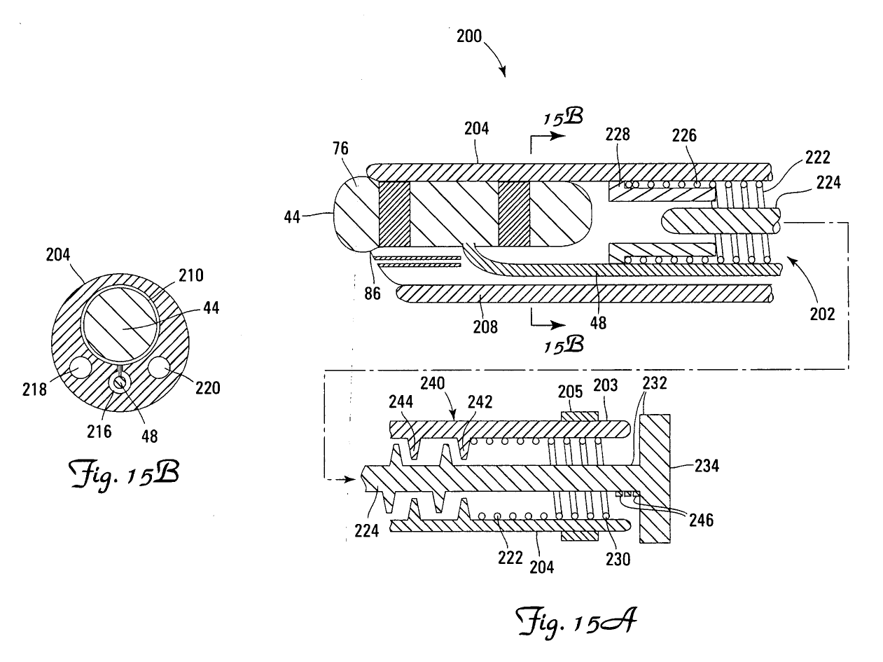

FIGS. 15A and 15B show a tool 200 for injecting the anchor 44 into the heart 14 according to another embodiment of the present invention. In the present embodiment, the tool 200 is adapted for automated injection of the anchor 44 into the heart 14. By automatic it is meant that the tool 200 allows the surgeon to inject the anchor 44 into the heart 14 with a pre-determined force and/or distance rather than by “feel” or estimation alone.

In the present embodiment, the tool 200 includes an injection mechanism 202 operably coupled to an elongated tool body 204 for holding and maneuvering the lead 22 into the heart 14.

The tool body 204 has a proximal end 203 adapted for grasping and manipulation by the surgeon, and may include a proximal handle or other grip 205. The tool body 204 has a distal end 208 provided with a nest 206 sized and shaped to securely hold at least a portion of the anchor 44. In one embodiment, as is shown in FIG. 15A, the nest 206 is sized so that a tool engaging shoulder 86 of the anchor 44 is inserted into the nest 206 while the first end 76 of the anchor 44 protrudes from the nest 206. Also shown in the FIG. 15A is an embodiment of the anchor 44 in which the first end 76 of the anchor 44 is blunted or made dull to facilitate dissection rather than cutting as the anchor 44 passes through the heart 14. In other embodiments, the tool 200 may include a cutting, needle-like or dissecting edge of its own and the anchor 44 may be fully received in the nest 106 and/or may lack a cutting or dissecting feature of its own.

The tool body 204 has a first lumen 210 extending proximally from the nest 206 in which the injection mechanism 202 is located. As is shown in FIG. 15B, the tool body 204 optionally has a second lumen 216 extending through the tool body 204 for holding the tether 48. A distal end of the second lumen 216 is slit open to the first lumen 210 to accommodate the connection between the tether 48 and the anchor 44.

The tool body 204 optionally includes additional or auxiliary lumens adapted for providing visualization or endoscopic tools, fluids, or other devices access to the heart 14. In the embodiment shown generally in FIGS. 15A and 15B, the tool body 204 includes first and second auxiliary lumens 218 and 220.

The injection mechanism 202 is positioned within the first lumen 210 proximal to the nest 206 and includes a coiled member 222 coupled to a plunger 224. In other embodiments, the coiled member 232 may be replaced with one or more elastic members capable of stretching and recoiling. A distal end 226 of the coiled member 222 is fixed to the tool body 204 proximal to the nest 206 with a coil ring 228. A proximal end 230 of the coiled member 222 is free to move within the first lumen 210 relative to the tool body 204. The plunger 224 extends through the center of the coiled member 222 and is coupled to the proximal end 223 of the coiled member 222. A proximal end 232 of the plunger 224 extends from the proximal end 203 of the tool body 204 and may have a gripping region 234 for easy manipulation by the surgeon.

The tool 200 is operable to inject the anchor 44 into the heart 14 as follows. First, the anchor 44 is loaded into the nest 206 with the tether 48 either extending outside of the tool body 204 or threaded into the second lumen 216. The surgeon then manipulates the proximal end 203 of the tool body 204 to advance the distal end 208 of the tool body 204 to the heart 14.

To inject the anchor 44 into the heart 14, the surgeon grasps the grip 234 at the proximal end 232 of the plunger 224 and withdraws the plunger 224 proximally, causing the coiled member 222 to stretch between the coil ring 228 and the proximal end 232 of the plunger 224, becoming tensioned or loaded. Upon a sudden release of the plunger 224, the coiled member 222 relaxes, sliding the plunger 224 through the tool body 204 distally through the lumen 210. The plunger 224 engages the anchor 44, ejecting the anchor 44 out of the nest 206 and injecting the anchor 44 into the heart 14 distal to the tool 200. The tool 200 is then withdrawn proximally over the tether 48. Doing so tensions the tether 48, causing the anchor 44 to deploy from the first configuration to the second configuration. The anchor 44 is now resistant to further proximal dislocation, so that as the tool 200 is withdrawn fully the tether 48 is extracted from the second lumen 216.

The size and tensioning force of the coiled member 222 may be chosen to provide sufficient injection force so as to cause the anchor 44 to puncture, for example, the epicardium 38, which is known to be a particularly tough tissue. It is believed that by puncturing the epicardium 38 in a single, somewhat forceful action, trauma to the epicardium 38 and to the heart 14 as a whole may be reduced. The shape of the first end of the anchor 44 may also be adapted to facilitate accessing tissues of the heart 14, as described previously. In addition, the diameter of the anchor 44 is such that the size of any opening through the epicardium 38 or other tissues of the heart 14 is reduced as well.

In one embodiment, the tool 200 includes an injection limiter 240 for preventing over injection of the anchor 44 into the heart 14. In the present embodiment, the injection limiter 240 is a cooperating flange structure located on the plunger 224 and the tool body 200 that functions to prevent excessive loading of the coiled member 222 and forward movement of the plunger 224 beyond a pre-determined location. In this manner, the plunger 224 may only be withdrawn as far proximally as a proximal flange 242 and may only advance distally as far as a distal flange 244.

In one embodiment, the tool 200 may also include gradations or other indicia 246 to guide the surgeon. The indicia 246 may relate to the length over which the plunger 224 is withdrawn proximally, potential injection force of the coiled member 222, the distance the anchor 44 is likely to be injected, or other factors that may help the surgeon to implant the anchor 44 in a chosen location.

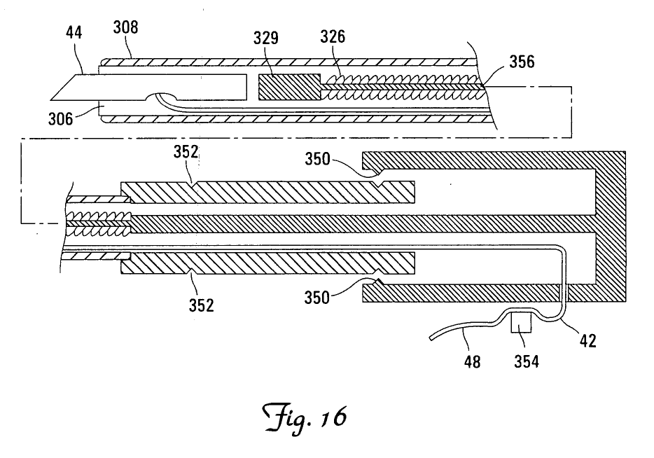

FIG. 16 shows a tool 300 for injecting the lead 22 into the heart according to another embodiment of the present invention. In the present embodiment, the tool 300 includes an injection mechanism 302 operably coupled to an elongated tool body 304 for holding a lead 22.

The tool body 304 has a proximal end 303 adapted for grasping and manipulation by the surgeon, and may include a proximal handle or other grip 305. The tool body 304 has a distal end 308 provided with a nest 306 sized and shaped to securely hold at least a portion of the anchor 44.

The tool body 304 has a first lumen 310 extending proximally from the nest 306 in which the injection mechanism 302 is located. The injection mechanism 302 is positioned within the first lumen 310 proximal to the nest 306 and includes a coiled member 322 coupled to a plunger 324. In contrast to the previous embodiment, the coiled member 322 is configured such that compression of the coiled member 322 causes the coiled member 322 to become loaded.

A proximal end 230 of the coiled member 322 is connected to the grip 305. The grip 305 is provided with cleats 350 for engaging detents 352 formed on the proximal end 303 of the tool body 304 and a hasp 354 for securing the proximal end 42 of the tether 48. Each detent 352 may be provided with indicia for indicating the distance separating them or indicating the distance or force through which the anchor 44 will be injected.

The distal end 326 of the coiled member 322 is free to slide within the first lumen 310 and terminates in a plunger 329. A tension wire 356 extends from the plunger 329 through the center of the coiled member 322 to the handle 305 and is sized to retain the coiled member 322 in a loaded state.

To implant the lead 22 into the heart 14, the anchor 44 is inserted into lumen 310 at the proximal end 303 of the tool body 304. The coiled member 322 is inserted into the first lumen 310 proximal to the anchor 44 and advanced distally to push the anchor 44 into the nest 306. The cleats 350 are inserted into a proximal detent 352 to secure the handle 305 to the tool body 304. The tether 48 is placed in the hasp 354 to secure the anchor 44 to the tool 100, but with a slight amount of slack.

The tool 300 is then inserted into the body and maneuvered to position the nest 306 at the desired implant location. The handle 305 is then pushed distally relative to the tool body 304 into a more distal cleat 352. The plunger 329 engages the anchor 44, ejecting the anchor 44 out of the nest 306 with sufficient force to inject the anchor 44 into the heart.

The spacing between the cleats 352 may be chosen to correspond to the injecting distance and may be regular or irregular. Furthermore, two or more distal cleats 352 may be provided to allow for different injection distances. Finally, the tether 48 is freed from the hasp 354 and the tool 300 is withdrawn proximally. The tether 48 is tensioned and the anchor 44 is deployed to the second configuration.

Because the injection mechanism 302 employs a flexible coiled member 322 rather than a solid plunger 329 to inject the anchor 44 from the nest 306, the tool 300 remains flexible and is operable to inject the anchor 44 even when the tool body 304 is bent or curved.

The previously described tools are operable in combination with a lead according to any embodiment of the present invention. Furthermore, the tool may be inserted into the myocardium 30 and the anchor 44 injected therefrom. Alternately, the tool may be positioned at or near a surface of the heart 14 such as the epicardium 38 and the anchor 44 injected into the myocardium 30 from that surface.

The lead 22 may be injected out of a tool into the endocardium 34 or epicardium 38 by transvenous, thoracoscopic, pericardioscopic or other transthoracic means. The above described procedure may be performed in a minimally invasive procedure such that the anchor 44 is injected into the heart 14 from the outside of the heart 14. The tool body 300 may be correspondingly sized and shaped to access the heart 14 from, for example, a sub-xiphoid insertion location. The tool body may be rigid or semi-rigid to provide sufficient support while advancing the tool towards the heart 14. Furthermore, the tool may be employed in conjunction with a dilator to provide access to the heart 14.

The anchor 44 may be injected into the heart 14 such that the anchor 44 passes through the epicardium 38 and is positioned within the myocardium 30. As shown in FIGS. 14A and 14B, the anchor 44 is injected into the myocardium-30 so that both electrodes 56 and 60 are disposed within the myocardium 30 for stimulating the myocardium 30. In one embodiment, the anchor 44 is injected a minimum distance I of one and a half times a length L of the anchor 44 into the myocardium 30.

In others embodiments, the anchor 44 may be injected still further through the myocardium 30 such that the anchor 44 traverses the endocardium 34 and is positioned within a chamber of the heart 14, such as the left ventricle. Upon tensioning of the tether 48, the anchor 44 is forced into contact with the endocardium 34 for endocardial stimulation. According to still other embodiments, the anchor 44 may be injected through the epicardium 38, the myocardium 30 and back out of the epicardium 38 for epicardial pacing.

The above described procedure may also be employed in a transvenous approach to the heart 14. In this embodiment, the tool body is correspondingly sized and shaped to allow the surgeon to manipulate the tool body through a series of vessels into the heart 14 from a more distal insertion location, such as the subclavian vein, into the coronary sinus. The tool may then be advanced into a lateral vein of the coronary sinus and the lead 22 injected through the epicardium 38. When employed in a transvenous approach to the heart 14, the tool body may be flexible. In one embodiment, the tool body is a catheter.

Therapeutically, the scope of the apparatus and method described herein may be employed for implantation of device specialized for resynchronization therapy and bradycardia therapy, and all heart chambers may be implanted with various embodiments. Furthermore, the present invention is not limited to implantation of a lead within the heart 14. Rather, the apparatus and methods generally described herein may also be used for neural applications.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. An injectable cardiac lead for stimulating a heart, the lead comprising:
an anchor having first and second electrically active areas spaced apart and electrically isolated from one another; and
a tether having a distal end mechanically coupled to the anchor and a proximal end adapted for coupling to a cardiac rhythm management device, wherein tether comprises first and second electrically conductive cables electrically isolated from one another and electrically coupled to the first and second electrically active areas, respectively.

2. The cardiac lead of claim 1 wherein the first and second electrically active areas comprise regions of the anchor made of an electrically conductive material.

3. The cardiac lead of claim 1 wherein the first and second electrically active areas comprise exposed portions of the cable coiled around the anchor.

4. The cardiac lead of claim 1 wherein the anchor is flexible.

5. The cardiac lead of claim 1 wherein the anchor is deployable from a first configuration adapted to traverse the myocardium to a second configuration adapted to resist traversing the myocardium.

6. The cardiac lead of claim 5 wherein the anchor further comprises wings deployable from a closed position in the first configuration to a open position in the second configuration.

7. The cardiac lead of claim 1 wherein the anchor has an outer diameter of from about 0.5 to about 0.833 mm.

8. The cardiac lead of claim 1 further comprising a detachment means for detaching the distal end of the tether from the anchor.

9. The cardiac lead of claim 1 further comprising a retention feature slidably engageable with the tether.

10. An injectable cardiac lead for stimulating a heart, the lead comprising:
an anchor having a first electrically active area; and
a tether having a distal end mechanically coupled to the anchor and a proximal end adapted for coupling to a cardiac rhythm management device, wherein the tether comprises a first electrically conductive cable electrically coupled to the first electrically active area, wherein an outer diameter of the tether from the proximal end to the distal end is substantially isodiametric.

11. The cardiac lead of claim 10 wherein the outer diameter of the tether is from about 0.267 to about 1 mm.

12. The cardiac lead of claim 11 wherein the outer diameter of the tether is about 0.5 mm.

13. The cardiac lead of claim 10 wherein the anchor further comprises a second electrically active area spaced apart from and electrically isolated from the first electrically active area.

14. The cardiac lead of claim 13 wherein the tether further comprises a second electrically conductive cable electrically coupled to the second electrically active area.

15. The cardiac lead of claim 10 wherein the outer diameter of the tether is less than about half of an outer diameter of the anchor.

16. A method of implanting a lead in a heart, the method comprising:
providing a lead, the lead having,
an anchor having first and second electrically active areas spaced apart and electrically isolated from one another; and
a tether having a distal end mechanically coupled to the anchor and a proximal end adapted for coupling to a cardiac rhythm management device, wherein tether comprises first and second electrically conductive cables electrically isolated from one another and electrically coupled to the first and second electrically active areas, respectively,
engaging the anchor to a distal end of an insertion tool;
advancing the distal end of the insertion tool to the heart;
injecting the anchor into a myocardium of the heart by ejecting the anchor from the insertion tool so that the first and second electrically active areas are located within the myocardium;
withdrawing the insertion tool proximally; and
deploying the anchor to a configuration adapted to resist migration by tensioning the tether cable.

17. The method of claim 16 wherein injecting the anchor into the heart includes actuating a spring-loaded injection mechanism on the insertion tool to engage the anchor.

18. The method of claim 16 wherein injecting the anchor into the heart further comprises injecting the anchor a pre-determined distance into the heart.

19. The method of claim 16 wherein injecting the anchor into the heart comprises injecting the anchor into the myocardium a minimum distance of approximately one and a half times a length of the anchor.
